(19) United States
(12) Patent Application Publication
(10) Pub. No.: US 2021/0177448 A1
(43) Pub. Date: Jun. 17, 2021

(54) SUCTION HEADS FOR VACUUM-ACTUATED SURGICAL GRASPERS

(71) Applicants: Chetan ELESWARPU, Oakland, CA (US); The Regents of the University of California, Oakland, CA (US)

(72) Inventors: Romain Roux, San Francisco, CA (US); Insoo Suh, San Francisco, CA (US); Isabelle Chumfong, San Francisco, CA (US); Chethan Eleswarpu, Oakland, CA (US); Sachin Rangarajan, Oakland, CA (US)

(21) Appl. No.: 17/269,960

(22) PCT Filed: Aug. 21, 2019
(86) PCT No.: PCT/US2019/047571

§ 371 Date: Feb. 19, 2021

Related U.S. Application Data
(60) Provisional application No. 62/720,471, filed on Aug. 21, 2018.

(51) Int. Cl. A61B 17/30; A61B 34/30

(52) U.S. Cl. CPC A61B 17/30; A61B 34/30; A61M 1/0088; A61B 2034/302; A61B 2017/306

(57) ABSTRACT

A suction head for a vacuum-actuated surgical grasper includes a proximal portion, a distal portion extending from the proximal portion, the distal portion being flared so as to have an outer dimension that increases from a proximal end of the distal portion to a distal end of the distal portion, and an inner passage extending through the suction head, the inner passage including a proximal portion that extends through the proximal portion of the suction head and a distal portion that extends through the distal portion of the suction head, wherein the distal portion of the inner passage is also flared so as to have an inner dimension that increases from the proximal end of the distal portion of the suction head to the distal end of the distal portion of the suction head, wherein the distal portion of the inner passage also includes internal ribs configured to grip tissue.

### CROSS-REFERENCE TO RELATED APPLICATION

This application claims priority to co-pending U.S. Provisional Application Ser. No. 62/720,471, filed Aug. 21, 2018, which is hereby incorporated by reference herein in its entirety.

### BACKGROUND

Mechanical surgical graspers have been used for decades in surgical procedures to manipulate (e.g., retract) patient tissues. Such graspers typically take the form of forceps that include a scissors-like handle that is used to open and close opposing jaws that can grip the tissue.

While such mechanical graspers are generally effective, their jaws can damage delicate tissues of a patient. Because of this, vacuum-actuated surgical graspers have been developed. Such graspers use a suction head to apply gentle suction to the tissue to grip it instead of opposed jaws, which can damage the delicate tissues. Needed, however, are suction heads for vacuum-actuated surgical graspers that enable tissue to be firmly gripped and manipulated while also being capable of passing through a narrow passage to the surgical site.

### BRIEF DESCRIPTION OF THE DRAWINGS

The present disclosure may be better understood with reference to the following figures. Matching reference numerals designate corresponding parts throughout the figures, which are not necessarily drawn to scale.

FIGS. 1A and 1B are side views of an embodiment of a vacuum-actuated surgical grasper, the grasper being shown with a suction head of the grasper not deployed and deployed, respectively.

FIG. 2 is a side view of a first embodiment of a suction head that can be used with the grasper of FIG. 1.

FIG. 3 a distal end view of the suction head of FIG. 2.

FIG. 4 is a cross-sectional side view of the suction head of FIG. 2.

FIG. 5 is a cross-sectional perspective view of the suction head of FIG. 2.

FIG. 6 is a cross-sectional side detail view of the suction head of FIG. 2.

FIG. 7 is partial cross-sectional side view of a second embodiment of a suction head that can be used with the surgical grasper of FIG. 1.

FIG. 8 is a schematic view of a robotic system that incorporates a vacuum-actuated surgical grasper that includes a suction head.

### DETAILED DESCRIPTION

As described above, needed are suction heads for vacuum-actuated surgical graspers that enable patient tissue to be firmly gripped and manipulated while also being capable of passing through a narrow passage to the surgical site. Examples of such suction heads are described in the disclosure that follows. In some embodiments, the suction heads have a flared configuration in which a lateral dimension (e.g., diameter) of the head increases from its proximal end to its distal end. In some embodiments, the suction heads are made of an elastomeric material that enables the head to be collapsed into a smaller size to facilitate its passage through a narrow lumen, such as a trocar. Once the suction head has passed through the lumen and reaches the surgical site (e.g., a site within the abdominal cavity), the suction head can be deployed and, therefore, enabled to expand into its natural flared shape. In such a case, the dimensions of the suction head can be substantially larger than the inner dimension (e.g., diameter) of the lumen through which the head is passed. The relatively large dimension of the suction head facilitates secure gripping of tissue.

In the following disclosure, various specific embodiments are described. It is to be understood that those embodiments are example implementations of the disclosed inventions and that alternative embodiments are possible, including hybrid embodiments that include aspects of separately disclosed embodiments. All such embodiments are intended to fall within the scope of this disclosure.

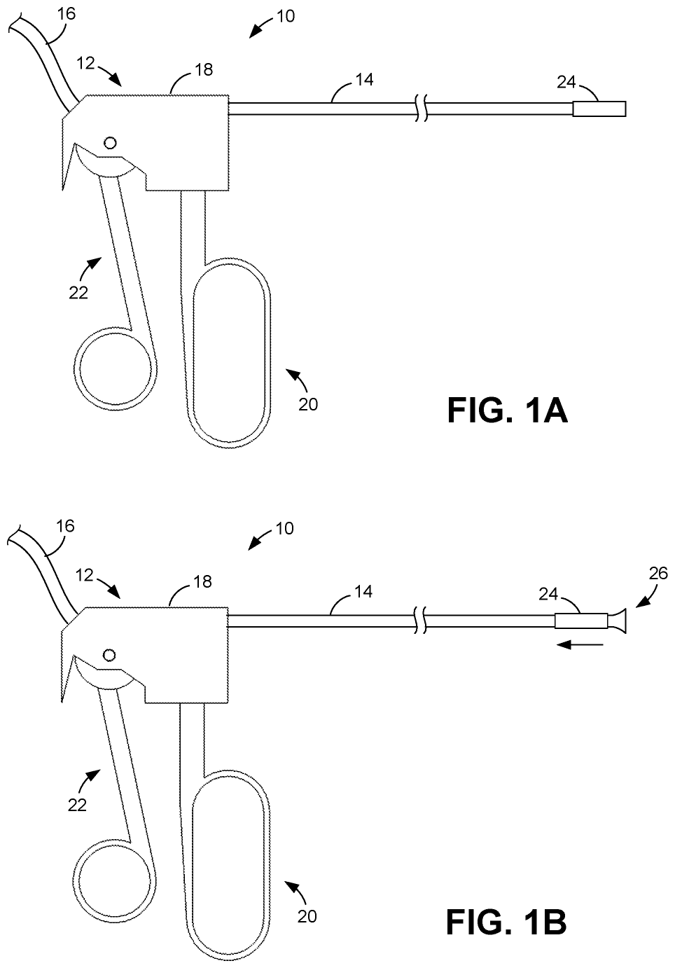

FIGS. 1A and 1B illustrate an example embodiment of a vacuum-actuated surgical grasper 10 that can be used in a surgical procedure, such as a laparoscopic procedure. This grasper 10 is intended for manual use by a human being. As shown in the figure, the grasper 10 generally comprises a body 12 and an elongated suction tube 14 that extends from the body. The grasper 10 is configured to connect to a vacuum source (not shown) via a vacuum tube 16 and deliver suction to the suction tube 14. In the illustrated embodiment, the body 12 includes a housing 18 from which the suction tube 14, a grip element 20, and an actuation lever 22 extend. With such a configuration, suction can be applied by a human user by moving the actuation lever 22 toward the grip element 20.

With further reference to FIGS. 1A and 1B, a sheath 24 is mounted to the distal end of the suction tube 14. In some embodiments, the sheath 24 comprises a generally rigid cylindrical element that is configured to contain and deploy a suction head 26 of the vacuum-actuated surgical grasper 10. In some embodiments, the sheath 24 has an outer dimension (e.g., outer diameter) of approximately 12 mm and an inner dimension (e.g., inner diameter) of approximately 11 mm. In FIG. 1A, the sheath 24 is shown in an extended position in which the suction head 26 is contained within the sheath in a collapsed orientation. In FIG. 1B, the sheath 24 is shown in a retracted position in which case the sheath has been linearly retracted along the suction tube 14 in the proximal direction (i.e., toward the body 12) so as to enable the suction head 26 to expand into its original natural flared shape. In some embodiments, such retraction is achieved using a wire or cable (not shown) that extends to the sheath 24 and is used to pull the sheath along the suction tube 14 in the proximal direction. Containment and deployment of the suction head 26 are facilitated by the material from which the suction head 26 is made. Specifically, the suction head 26 is made of a biocompatible elastomeric material, such as a medical-grade silicone material, that enables the suction head to be collapsed within the sheath 24 and to spring back into its original natural expanded (i.e., flared) state shown in FIG. 1B once the sheath is pulled back.

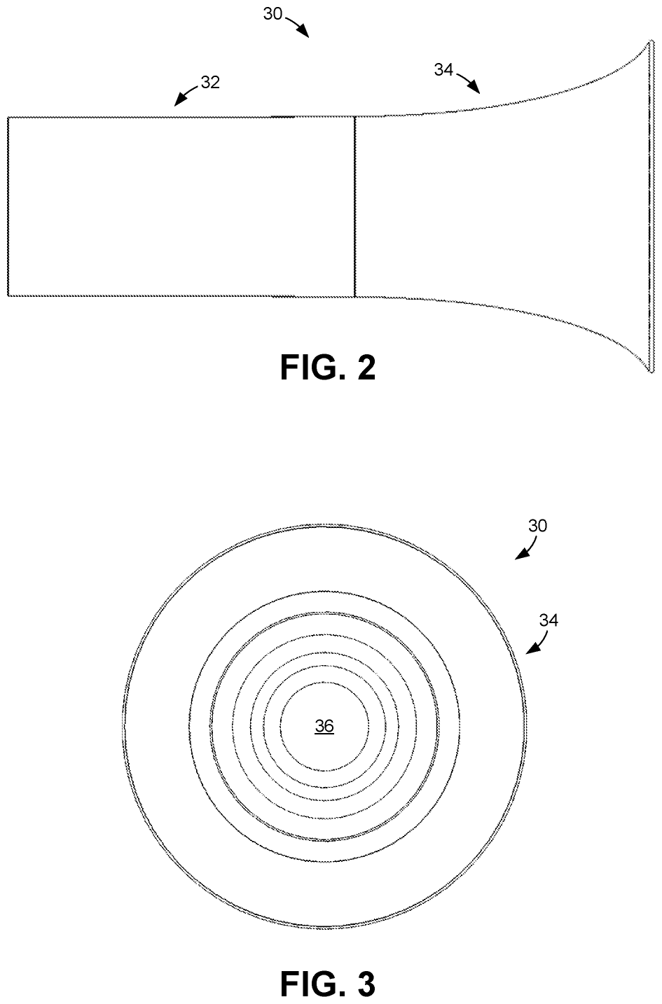

FIGS. 2-6 illustrate a first embodiment of the suction head, which is identified by reference numeral 30. With reference first to FIG. 2, the suction head 30 generally comprises a proximal portion 32 and a distal portion 34 that extends from the proximal portion. In some embodiments, both portions 32 and 34 are made from the same piece of material such that the suction head 30 has a unitary construction. As mentioned above, the suction head 30 can be made of an elastomeric material, such as a medical-grade silicone material. In some embodiments, the elastomeric material has a Shore A hardness of approximately 40A to 100A (e.g., Shore 60A).

The proximal portion 32 is generally cylindrical and has a constant outer dimension (e.g., outer diameter) while the distal portion 34 has a flared shape in which the outer dimension (e.g., outer diameter) of the distal portion gradually increases from its proximal end to its distal end. In cases in which the suction head 30 is generally circular in cross-section, the suction head can be described as being generally trumpet shaped in that it incorporates a flared end similar to that of the “bell” of a trumpet, which can be said to form the shape of a cone that has a geometrically (i.e., nonlinearly) increasing diameter from its proximal end to its distal end. In some embodiments, the proximal portion 32 has a constant outer diameter of approximately 9 mm, while the distal portion 34 has an outer diameter that is equal to that of the proximal portion (e.g., approximately 9 mm) at its proximal end but geometrically increases to approximately 17 mm at its distal end.

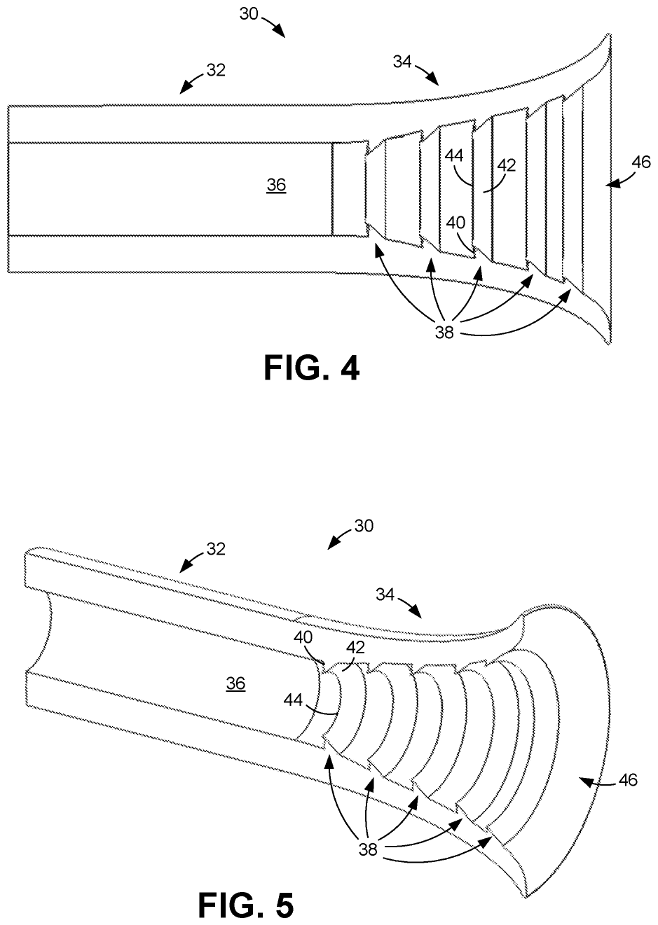

With reference to FIGS. 4 and 5, the suction head 30 forms an elongated inner passage 36 that extends along the length of the head from its proximal end to its distal end. This passage 36 is placed in fluid communication with an inner passage of the suction tube 14 when the head 30 is mounted to the distal end of the tube. In some embodiments, the distal end of the suction tube 14 is received within the inner passage 36 of the proximal portion 32 with a friction fit. As shown in FIGS. 4 and 5, the inner passage 36 can have a constant dimension (e.g., inner diameter) within the proximal portion 32 and, like the outer surface of the head 30, a dimension (e.g., inner diameter) that gradually (geometrically) increases from the proximal end of the distal portion 34 to the distal end of the distal portion. In some embodiments, the passage 36 has an inner diameter of approximately 5 mm within the proximal portion 32 and an inner diameter that increases from approximately 5 mm to approximately 17 mm within the distal portion 34. As can be appreciated from FIG. 4, the thickness of the walls of the distal portion 34 can gradually decrease from its proximal end to its distal end.

With reference next to FIGS. 4-6, the distal portion of the inner passage 36, which is contained within the distal portion 34 of the suction head 30, comprises multiple internal continuous annular ribs 38 that facilitate gripping of the tissue that is drawn into the inner passage when the vacuum-actuated surgical grasper 10 is used. In the illustrated embodiment, the suction head 30 comprises 5 such ribs 38, which are spaced from each other along the longitudinal direction of the distal portion of the inner passage 36. In some embodiments, the ribs 38 increase in size from the distal end to the proximal end of the distal portion 34 of the head 30.

As shown in FIG. 4, each rib 38 is generally triangular in cross-section and includes an arcuate proximal surface 40 that generally faces the proximal end of the suction head 30, an arcuate distal surface 42 that generally faces the distal end of the head, and an arcuate edge 44 that is formed where the proximal and distal surfaces meet. As is further shown in the figure, the distal surface 42 of each rib 38 is larger (i.e., extends a farther distance from the wall of the inner passage 36) than the proximal surface 40. This configuration results in each rib 38 having a swept orientation in which each rib is angled backward toward the proximal end of the suction head 30 and the arcuate edge 44 of each rib (and, therefore, the tip of the triangular cross-section) faces that end, away from an inlet 46 of the head in which tissue is drawn. This swept orientation increases the gripping force that can be applied to the tissue by the head 30.

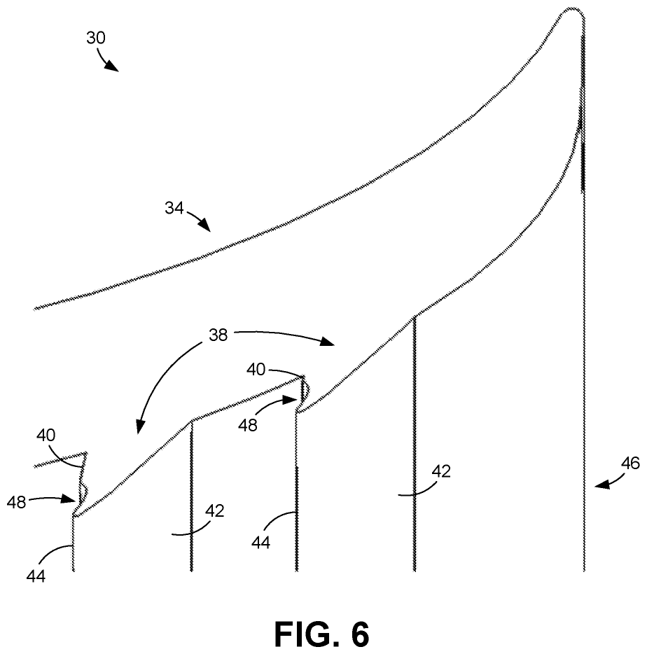

Referring next to FIG. 6, each rib 38 can further comprise concave arcuate groove 48 that is formed in the proximal surface 40 of the rib and, therefore, also faces the proximal end of the suction head 30. When provided, the grooves 48 reduce the amount of material that forms the arcuate edges 44 of the ribs 38, which enables the ribs to flex backward toward the inlet 46 of the suction head 30. This flexure further enhances the grip of the suction head 30 on the tissue, particularly when the head is used to retract tissue.

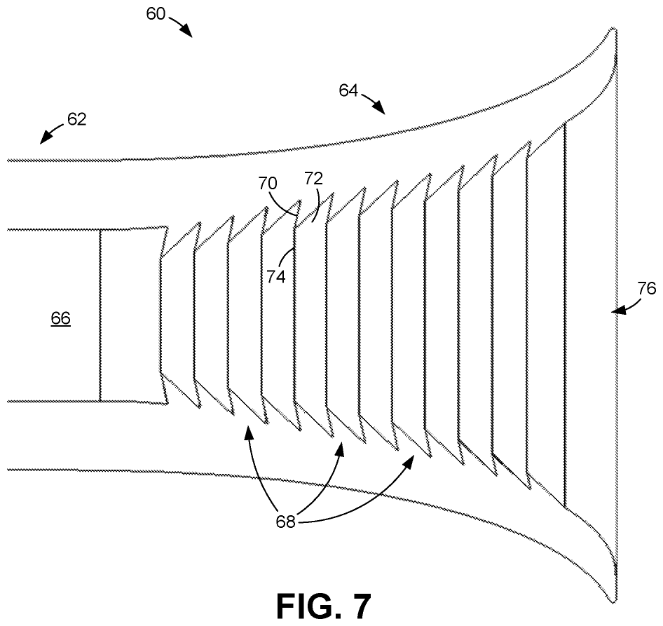

FIG. 7 illustrates a further embodiment of a suction head 60. The suction head 60 can have a configuration similar to that of the suction head 30. Accordingly, the suction head 60 can comprise a cylindrical proximal portion 62 and a flared distal portion 64. The suction head 60 can also be made of an elastomeric material, such as a medical-grade silicone material.

Also like the suction head 30, the suction head 60 includes an elongated inner passage 66 that extends along the length of the head from its proximal end to its distal end. The inner passage 66 includes multiple internal continuous annular ribs 68 that facilitate gripping of tissue that is drawn into the inner passage when the vacuum-actuated surgical grasper 10 is used. In the illustrated embodiment, the suction head 60 comprises 12 such ribs 68, each positioned immediately adjacent to each other along the length of the inner passage 66 within the distal portion 64 of the head. In some embodiments, the ribs 68 increase in size from the distal end to the proximal end of the distal portion 64 of the head 60.

As shown in FIG. 7, each rib 68 is generally triangular in cross-section and includes an arcuate proximal surface 70 that generally faces the proximal end of the suction head 60, an arcuate distal surface 72 that generally faces the distal end of the head, and a sharp arcuate edge 74 that is formed where the proximal and distal surfaces meet. As with the other embodiment, the distal surface 72 of each rib 68 is larger (i.e., extends a farther distance from the wall of the inner passage 66) than the proximal surface 70. This configuration results in each rib 68 also having a swept orientation in which each rib is angled backward toward the proximal end of the suction head 60 and the arcuate edge 74 of each rib (and, therefore, the tip of the triangle cross-section) faces that end, away from an inlet 76 of the head in which tissue is drawn into the inner passage 36. Unlike the ribs 38 of the suction head 30, however, the ribs 68 of the suction head 60 do not include an arcuate groove. Because of this, the suction head 60 is easier and less expensive to manufacture.

Irrespective of the particular configuration of the suction head, the vacuum-actuated surgical grasper 10 (FIG. 1) can be used to grasp patient tissues to manipulate (e.g., refract) them. During such use, a vacuum source can be connected to the vacuum tube 16 so that a fluid, such as air or another gas, can be drawn through the suction head (e.g., suction head 26), the suction tube 14, and the body 12. When the suction head is placed in contact with tissue while the vacuum is applied, a portion of the tissue is drawn into the suction head and, therefore, is securely gripped by the head. In some embodiments, tissues can be gripped with a force of approximately 10 N, which is comparable to conventional jawed graspers. Unlike jawed graspers, however, damage to the tissue is avoided as no sharp edges or hard materials come into contact with the tissue.

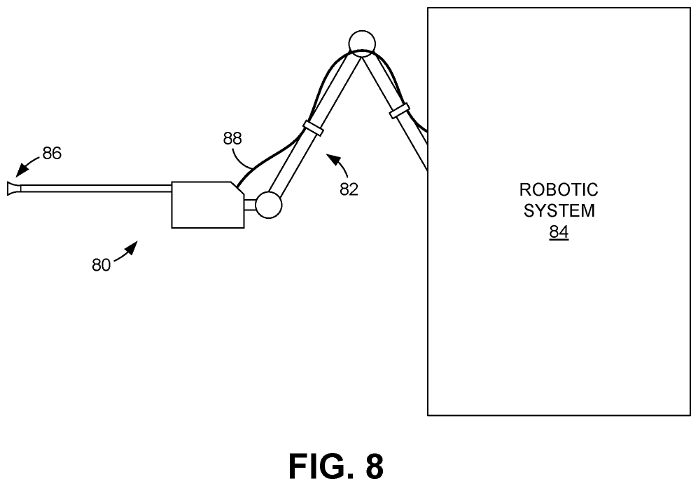

Although a vacuum-actuated surgical grasper has been described and illustrated that is configured for manual operation by a human user, it is noted that a similar vacuum-actuated surgical grasper can be used in a robotics context. This is schematically illustrated in FIG. 8. As shown in this figure, a vacuum-actuated surgical grasper 80 is attached to the end of a robotic manipulator 82 of a robotic system 84. In such a case, the surgical grasper 80 functions as an end effector of the system 84 and its position and orientation can be robotically controlled using the robotic manipulator 82. Once the suction head 86 is positioned and oriented as desired, for example, so that the head is placed in contact with tissue that is to be manipulated, suction can be applied to the head via a vacuum tube 88 that extends either along or within the robotic manipulator to a vacuum source (not shown). Accordingly, instead of moving and applying suction manually as with the embodiment shown in FIGS. 1A and 1B, such moving and applying suction is automated by the robotic system 84.

Claimed are:

1. A suction head for a vacuum-actuated surgical grasper, the suction head comprising:
a proximal portion;
a distal portion extending from the proximal portion, the distal portion being flared so as to have an outer dimension that increases from a proximal end of the distal portion to a distal end of the distal portion; and
an inner passage extending through the suction head, the inner passage including a proximal portion that extends through the proximal portion of the suction head and a distal portion that extends through the distal portion of the suction head, wherein the distal portion of the inner passage is also flared so as to have an inner dimension that increases from the proximal end of the distal portion of the suction head to the distal end of the distal portion of the suction head, wherein the distal portion of the inner passage also includes internal ribs configured to grip tissue.

2. The suction head of claim 1, wherein the suction head is made of a biocompatible elastomeric material.

3. The suction head of claim 2, wherein the biocompatible elastomeric material is a medical-grade silicone material.

4. The suction head of claim 2, wherein the biocompatible elastomeric material has a Shore A hardness of approximately 40A to 100A.

5. The suction head of claim 1, wherein the proximal portion of the suction head is cylindrical and has a constant outer diameter.

6. The suction head of claim 5, wherein the outer diameter is approximately 9 millimeters.

7. The suction head of claim 1, wherein the distal portion of the suction head has an outer diameter that is at a minimum at the proximal end of the distal portion of the suction head and at a maximum at the distal end of the distal portion of the suction head.

8. The suction head of claim 7, wherein the minimum outer diameter is approximately 9 millimeters and the maximum outer diameter is approximately 17 millimeters.

9. The suction head of claim 1, wherein the distal portion of the suction head is flared in a manner in which its outer diameter geometrically increases from the proximal end of the distal portion of the suction head to a distal end of the distal portion of the suction head.

10. The suction head of claim 1, wherein the distal portion of the suction head is configured to collapse and retract into a sheath having an inner diameter of no greater than 12 millimeters.

11. The suction head of claim 1, wherein the internal ribs are arcuate and continuous.

12. The suction head of claim 11, wherein the internal ribs have generally triangular cross-sections.

13. The suction head of claim 12, wherein the internal ribs have swept orientations in which they are angled back toward the proximal portion of the suction head.

14. The suction head of claim 13, wherein the internal ribs each have an arcuate proximal surface, an arcuate distal surface, and an arcuate edge formed where the proximal and distal surfaces meet.

15. The suction head of claim 14, wherein the arcuate proximal surfaces of the internal ribs each include a concave arcuate groove near the arcuate edge that enables the internal rib to flex.

16. A vacuum-actuated surgical grasper comprising:
a body;
an elongated suction tube extending from the body; and
a suction head connected to and in fluid communication with the suction tube, the suction head comprising:
a proximal portion,
a distal portion extending from the proximal portion, the distal portion being flared so as to have an outer dimension that increases from a proximal end of the distal portion to a distal end of the distal portion, and
an inner passage extending through the suction head, the inner passage including a proximal portion that extends through the proximal portion of the suction head and a distal portion that extends through the distal portion of the suction head, wherein the distal portion of the inner passage is also flared so as to have an inner dimension that increases from the proximal end of the distal portion of the suction head to the distal end of the distal portion of the suction head, wherein the distal portion of the inner passage also includes internal ribs configured to grip tissue.

17. The surgical grasper of claim 16, wherein the suction head is made of a biocompatible elastomeric material.

18. The surgical grasper of claim 16, wherein the distal portion of the suction head is flared in a manner in which its outer diameter geometrically increases from the proximal end of the distal portion of the suction head to a distal end of the distal portion of the suction head.

19. The surgical grasper of claim 16, wherein the internal ribs are arcuate and continuous, and have generally triangular cross-sections.

20. The surgical grasper of claim 19, wherein the internal ribs have swept orientations in which they are angled back toward the proximal portion of the suction head.

21. The surgical grasper of claim 20, wherein the internal ribs each have an arcuate proximal surface, an arcuate distal surface, and an arcuate edge formed where the proximal and distal surfaces meet.

22. The surgical grasper of claim 21, wherein the arcuate proximal surfaces of the internal ribs each include a concave arcuate groove near the arcuate edge that enables the internal rib to flex.

23. The surgical grasper of claim 16, further comprising a retractable sheath provided on the suction tube that is configured to surround the suction head in a collapsed state to enable the suction head to pass through a lumen.

24. The surgical grasper of claim 16, wherein the surgical grasper is configured for manual operation by a human user.

25. The surgical grasper of claim 16, wherein the surgical grasper is configured for automated operation by a robotic system.
